(19) United States
(12) Patent Application Publication
(10) Pub. No.: US 2026/0184696 A1
(43) Pub. Date: Jul. 2, 2026

(54) A PROCESS FOR THE PREPARATION OF LEVOKETOCONAZOLE

(71) Applicant: PIRAMAL PHARMA LIMITED, Mumbai (IN)

(72) Inventors: Ashutosh Jagtap, Mumbai (IN); Changdev Raut, Mumbai (IN); Yogeshwar Sabale, Mumbai (IN); Suyog Marathe, Mumbai (IN); Mihir Kachhia, Mumbai (IN)

(73) Assignee: PIRAMAL PHARMA LIMITED, Mumbai (IN)

(21) Appl. No.: 19/129,945

(22) PCT Filed: Nov. 10, 2023
(86) PCT No.: PCT/IB2023/061372

§ 371 Date: May 14, 2025

(30) Foreign Application Priority Data
Nov. 14, 2022 (IN) 202221065074

(51) Int. Cl. C07D 405/06

(52) U.S. Cl. CPC C07D 405/06

(57) ABSTRACT

A process for the preparation of Levoketoconazole (1) includes reacting ketoconazole with (1S)-(+) camphor sulphonic acid in a solvent to give (1S)-(+) camphor sulphonic acid salt of Levoketoconazole; treating Levoketoconazole Camphor sulphonic acid with an aqueous base to give crude Levoketoconazole (1); and isolating and purifying crude Levoketoconazole (1) to yield pure Levoketoconazole (1). A process for the preparation of crystalline anhydrous Levoketoconazole (1) is also included.

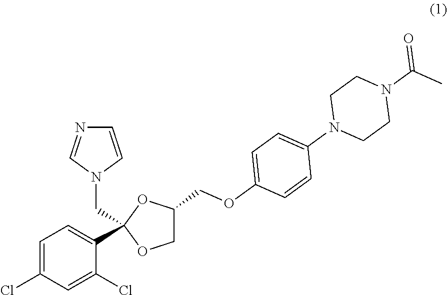 [SMILES: CC(=O)N1CCN(c2ccc(OC[C@@H]3CO[C@@](Cn4ccnc4)(c4ccc(Cl)cc4Cl)O3)cc2)CC1]

### FIELD OF THE INVENTION

The present invention relates to a process for the preparation of Levoketoconazole (1).

### BACKGROUND OF THE INVENTION

The following discussion of the prior art is intended to present the invention in an appropriate technical context and allows its significance to be properly appreciated. Unless clearly indicated to the contrary, reference to any prior art in this specification should not be construed as an expressed or implied admission that such art is widely known or forms part of common general knowledge in the field.

Levoketoconazole (1) is one of two enantiomeric forms of racemic Ketoconazole (2) that strongly inhibits multiple steroidogenic enzymes and is used for the symptomatic treatment of endogenous Cushing's syndrome. Levoketoconazole (1) was approved by the FDA on 30 Dec. 2021 and is currently marketed under the registered trademark RECORLEV® by Xeris Pharmaceuticals, Inc.

Levoketoconazole (1) is chemically known as 2S, 4R cis-1-acetyl-4-[4-[[2-(2,4-dichlorophenyl)-2-(1H-imidazol-1-ylmethyl)-1,3-dioxolan-4-yl]methoxyl]phenyl]piperazine, as shown below a Compound of Formula (1). Levoketoconazole is the levorotatory or (2S, 4R) enantiomer of Ketoconazole (2).

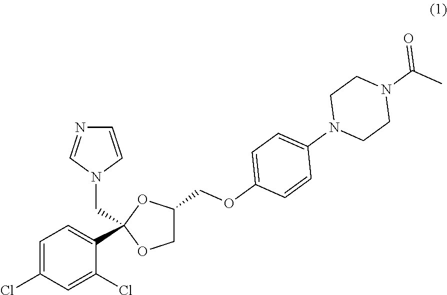 [SMILES: CC(=O)N1CCN(c2ccc(OC[C@@H]3CO[C@@](Cn4ccnc4)(c4ccc(Cl)cc4Cl)O3)cc2)CC1]

Levoketoconazole (1) viz. (2S, 4R) enantiomer of Ketoconazole has been disclosed for the first time in Journal of Medicinal Chemistry (1992), 35 (15), 2818-25. This research article describes the synthesis of Levoketoconazole on page no. 2824 in which Bromo compound of formula (3) is reacted with imidazole in presence of anhydrous K₂CO₃ in DMF at reflux temperature to give Levoketoconazole which is then subjected to flash chromatography to give pure Levoketoconazole. The schematic representation is shown below:

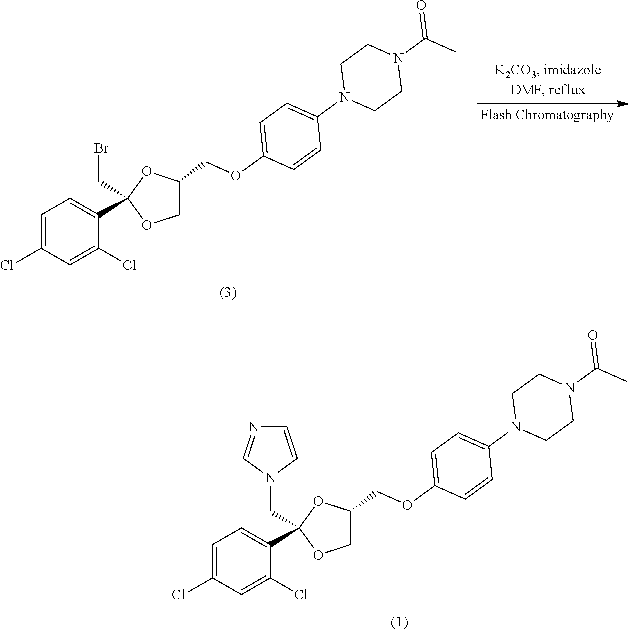 [SMILES: CC(=O)N1CCN(c2ccc(OC[C@@H]3CO[C@@](CBr)(c4ccc(Cl)cc4Cl)O3)cc2)CC1 ; CC(=O)N1CCN(c2ccc(OC[C@@H]3CO[C@@](Cn4ccnc4)(c4ccc(Cl)cc4Cl)O3)cc2)CC1]

WO1996029325A1 reported the preparation of Levoketoconazole (1) in example 17 by treating mesylate protected compound (4) with 1-[4-(4-Hydroxy-phenyl) piperazin-1-yl]ethanone in presence of sodium hydride in anhydrous DMSO with heating at 80° C. for 4 hours. The process as shown in Scheme-II below:

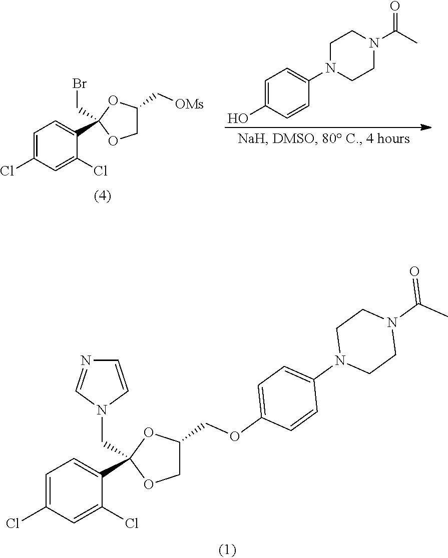 [SMILES: CS(=O)(=O)OC[C@@H]1CO[C@@](CBr)(c2ccc(Cl)cc2Cl)O1 ; CC(=O)N1CCN(c2ccc(O)cc2)CC1 ; CC(=O)N1CCN(c2ccc(OC[C@@H]3CO[C@@](Cn4ccnc4)(c4ccc(Cl)cc4Cl)O3)cc2)CC1]

J Pharm Sci 1999, 88 (6): 599 states preparation of Levoketoconazole by performing resolution of Ketoconazole by using (1S)-(+) camphor sulphonic acid [CSA]. However, no detailed description is given in the journal.

The inventors of the present invention found that very limited literature is available which discloses the preparation of Levoketoconazole, and the reported methods mentioned suffer from drawbacks such as low chiral purity of the final product and lack of robustness of the process.

Hence, there is always a need for an efficient process for the preparation of pharmaceutically active products. Therefore, the inventors of the present invention have developed the process, which gives good chiral purity of the final product and overall process is more user-friendly and much suitable for large scale reactions.

So, present inventors have accordingly developed a simple, cost-effective, production friendly process which yields Levoketoconazole with desired chiral purity. In the process of the instant invention, there is a significant overall advancement in chiral purity and work-up procedure as against prior art methods, and the process is safe and easy to operate at large scale.

### SUMMARY OF THE INVENTION

The problem addressed by the present invention is therefore that of providing a better process for preparing of Levoketoconazole (1), which permits to avoid above drawbacks reported with reference to the known prior art.

This problem is solved by a process for the preparation of Levoketoconazole (1) which involves resolution of ketoconazole by using (1S)-(+) camphor sulphonic acid in a mixture of solvents.

### BRIEF DESCRIPTION OF DRAWINGS

FIG. 1: X-ray diffraction (XRD) pattern of Levoketoconazole (1), prepared according to Example 1

FIG. 2: DSC thermogram of Levoketoconazole (1), prepared according to example 1.

FIG. 3: TGA (Thermal gravimetric analysis) of Levoketoconazole (1), prepared according to example 1.

### DETAILED DESCRIPTION OF THE INVENTION

Accordingly, the present invention relates to a process for the preparation of Levoketoconazole (1),

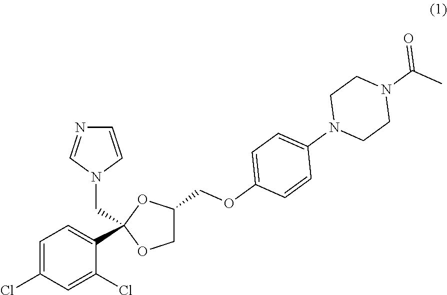 [SMILES: CC(=O)N1CCN(c2ccc(OC[C@@H]3CO[C@@](Cn4ccnc4)(c4ccc(Cl)cc4Cl)O3)cc2)CC1]

comprising;
(a) reacting Ketoconazole (2) with (1S)-(+) camphor sulphonic acid in a solvent to give (1S)-(+) camphor sulphonic acid salt of Levoketoconazole (1a);

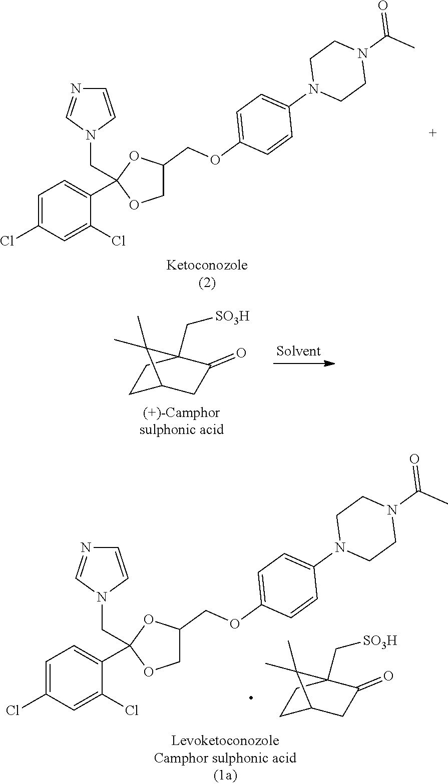

(b) treating Levoketoconazole Camphor sulphonic acid compound of formula (1a) with an aqueous base to give Levoketoconazole (1); and

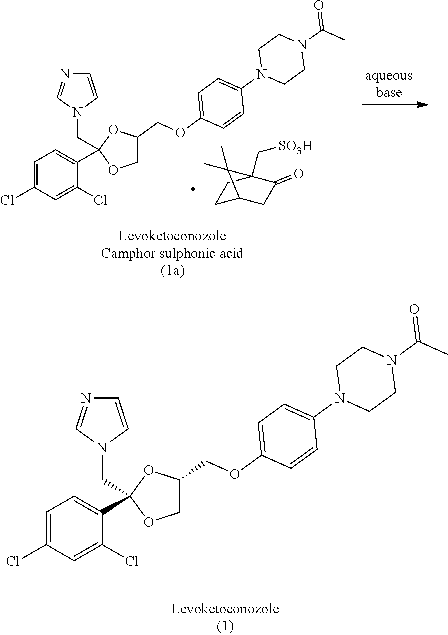

(c) isolating and purifying crude Levoketoconazole (1) obtained in step (b) to yield pure Levoketoconazole (1).

The present invention also relates to the process, wherein step (b) can be carried out without isolating the compound of formula (1a).

The solvent used in step (a) is an ether solvent selected from tetrahydrofuran, cyclopentyl methyl ether, 2-methyltetrahydrofuran, diethyl ether, dioxane, 1,4-dioxane, 1,2-dioxane or 1,3-dioxane; an alcoholic solvent selected from methanol, ethanol, isopropanol, t-amyl alcohol, t-butyl alcohol or hexanol; a halogenated solvent selected from dichloromethane, 4-bromotoluene, diiodomethane, carbon tetrachloride, chlorobenzene or chloroform; a ketone solvent selected from acetone, propanone, methyl ethyl ketone or methyl isobutyl ketone; an aprotic solvent selected from acetonitrile, N,N-dimethyl formamide (DMF), N,N-dimethyl acetamide, dimethyl sulfoxide (DMSO) or N-methylpyrrolidone (NMP); an aromatic solvent selected from toluene, xylene or benzene; water or a mixture thereof.

The base used in step (b) is an alkali metal hydroxide selected from lithium hydroxide, sodium hydroxide or potassium hydroxide; alkali metal carbonate selected from lithium carbonate, sodium carbonate, potassium carbonate or cesium carbonate; alkali metal bicarbonates selected from sodium bicarbonate or potassium bicarbonate; alkali metal alkoxides selected from sodium methoxide or potassium methoxide, sodium ethoxide or potassium ethoxide or potassium tert-butoxide, or organic amines selected from triethylamine, diisopropylethylamine, pyridine, 1,8-diazabicyclo[5.4.0]undec-7-ene (DBU) or 1,5-diazabicyclo[4.3.0]non-5-ene (DBN).

The base used in step (b) is selected from aqueous base selected from sodium hydroxide, potassium hydroxide, barium hydroxide, lithium hydroxide, sodium methoxide, sodium ethoxide, potassium tert butoxide, potassium carbonate, sodium carbonate, cesium carbonate, sodium bicarbonate, potassium bicarbonate.

The whole synthetic scheme of preparation of Levoketoconazole (1) according to the present invention can be represented as below:

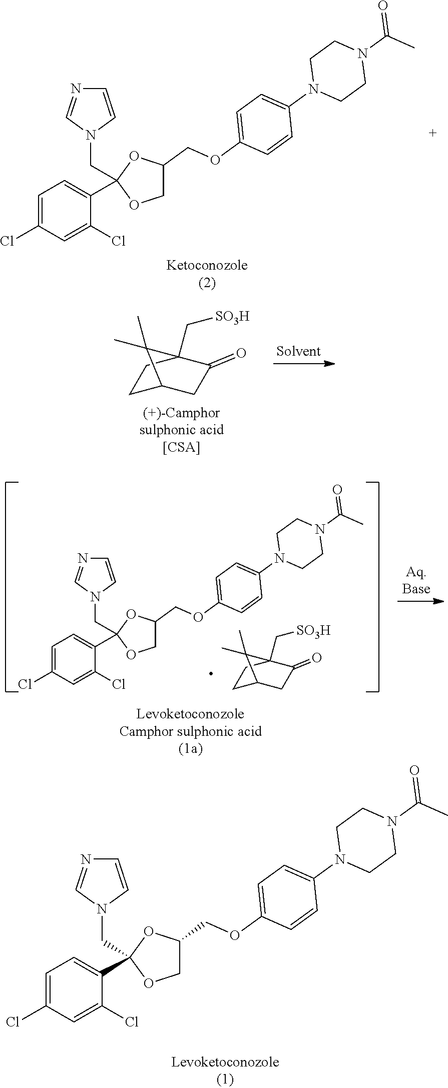

According to the present invention, isolation followed by purification of Levoketoconazole (1) from reaction mass of step (b) comprises the steps of:
(i) concentrating the reaction mixture of step (b) to obtain the residue;
(ii) crystallizing obtained residue of step (i) in a solvent; and
(iii) filtering the precipitate of step (ii) and drying the obtained precipitate to obtain crystalline solid of Levoketoconazole (1).

The solvent used in step (ii) is an alcoholic solvent selected from methanol, ethanol, isopropanol, t-amyl alcohol, t-butyl alcohol or hexanol.

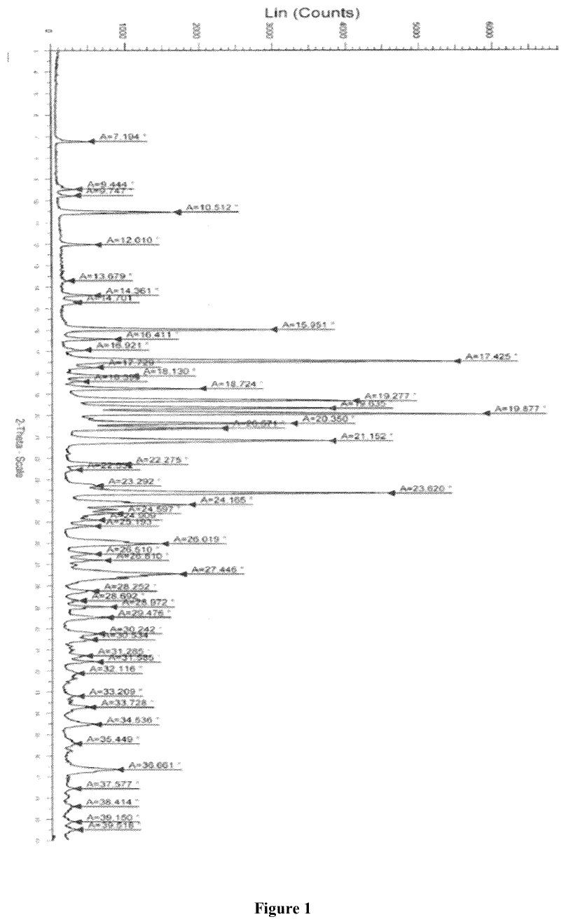

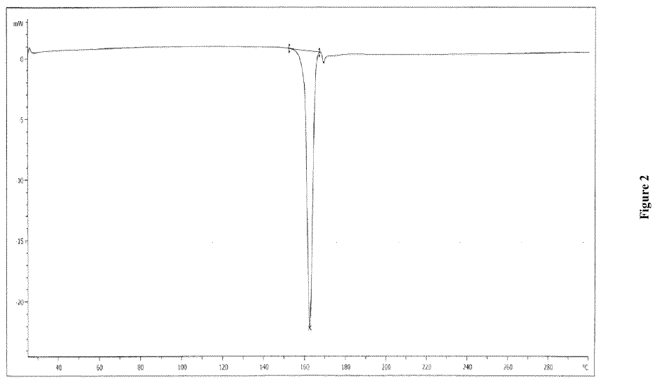

According to the invention, there is provided crystalline Levoketoconazole (1). FIG. 2 illustrates X-ray powder diffraction (XPRD) pattern of form Levoketoconazole (1), prepared according to example 1. The X-ray diffractogram was measured on Bruker Axe, DS advance Power X-ray Diffractometer with Cu K alpha-1 Radiation source having the wavelength 1.541 A⁰. The crystalline Levoketoconazole (1) of the present invention is characterized by its powder X-Ray diffraction pattern having the peaks at 10.51, 15.95, 17.42, 19.27, 19.87, 21.15 and 23.62±0.2 degrees of 2-theta as shown in FIG. 1.

FIG. 2 of the present invention illustrates the Differential scanning calorimetry (DSC) thermogram of crystalline Levoketoconazole (1), prepared according to example 1, it exhibits endothermic peak between 159° C. to 162° C.

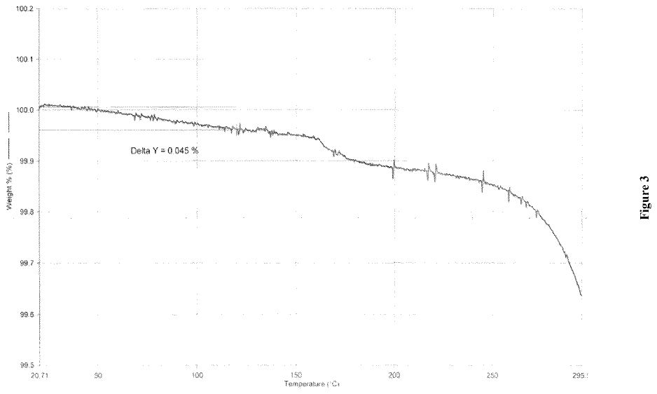

FIG. 3 of the present invention illustrates the thermogravimetric analysis (TGA) of crystalline Levoketoconazole (1), prepared according to example 1, which indicates that the obtained Levoketoconazole (1) is anhydrous in nature.

Thus, the present invention has several advantages over previous methods reported in the literature which include:
(i) obtaining a chiral purity more than 99.50%;
(ii) robustness of the developed process.

Thereby, the practicability of the reaction is greatly enhanced both at the laboratory scale and the industrial scale. The present invention results into chiral purity of at least 99.9% by HPLC, thereby, making the process efficient, economic and industrially viable.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example 1

### Preparation of Levoketoconazole (1)

Charged Ketoconazole (0.50 kg, 0.94 mol, 1 eq) in a reactor followed by the addition of Methyl Ethyl Ketone (0.375 L) and Iso Propyl Alcohol (0.375 L). After mixing it well, (1S)-(+)-Camphor Sulphonic Acid was charged (0.186 kg, 0.8 mol, 0.85 eq). The reaction mixture was then cooled below 10° C. and stirred for 3-6 h. The reaction mass was then filtered to yield Levoketoconazole camphor sulphonic acid (LKTN-CSA) salt. The LKTN-CSA salt wet cake was charged to the reactor. A mixture of 1.5 L of Isopropyl alcohol and 0.45 L of methanol was charged to the reactor and the reaction mixture was heated over 65° C. Then the reaction mixture was cooled to room temperature, stirred for 4-6 h and filtered to yield purified salt. The purified salt was charged to the reactor and 0.925 L of 1.0% aqueous sodium carbonate solution and 1.85 L of methylene dichloride (MDC) were charged into the reactor and the mixture was stirred. The layers were separated, and the MDC layer was washed with 0.925 L of dimeralized water twice. 5 g activated charcoal was charged to the MDC layer in the reactor and stirred for 0.5 h. The MDC layer was then filtered through a Hyflo bed using 0.45 micron filter. Further, the MDC layer was concentrated. 0.650 L of isopropyl alcohol was charged into the crude in the reactor and heated till it became a clear solution. The reaction mixture was then cooled to 0-5° C. and filtered to yield the final product. Levoketoconazole wet cake was dried in vacuum tray dryer to yield Levoketoconazole (125 g, 50% yield, purity >99.85).

The XPRD of the obtained compound of formula (1) is given in FIG. 1.

The DSC of the obtained compound of formula (1) is given in FIG. 2, which indicates endotherm between 159° C. to 162° C.

The TGA of the obtained compound of formula (1) is given in FIG. 3, which indicates anhydrous nature of the compound.

1. A process for the preparation of Levoketoconazole (1), 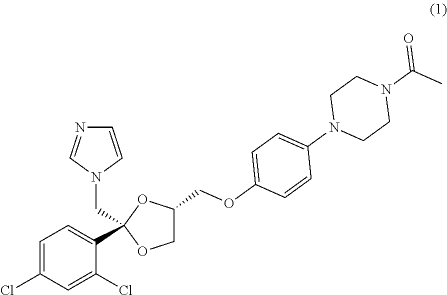 [SMILES: CC(=O)N1CCN(c2ccc(OC[C@@H]3CO[C@@](Cn4ccnc4)(c4ccc(Cl)cc4Cl)O3)cc2)CC1]
the process comprising;
(a) reacting ketoconazole (2) with (1S)-(+) camphor sulphonic acid in a solvent to produce (1S)-(+) camphor sulphonic acid salt of Levoketoconazole (1a); 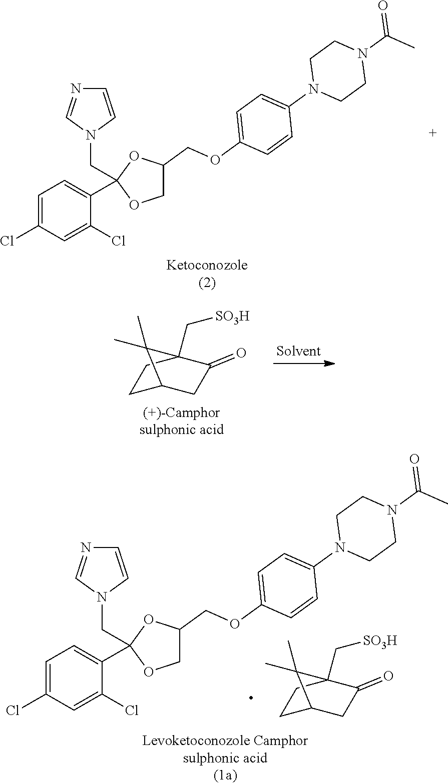
(b) treating the (1S)-(+) camphor sulphonic acid salt of Levoketoconazole (1a) with an aqueous base to produce crude Levoketoconazole (1); and 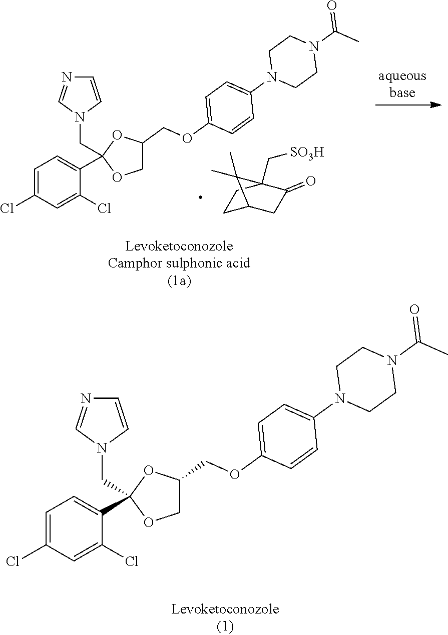
(c) isolating and purifying crude Levoketoconazole (1) obtained in step (b) to yield pure Levoketoconazole (1).

2. The process as claimed in claim 1, wherein the solvent is an ether solvent selected from the group consisting of tetrahydrofuran, cyclopentyl methyl ether, 2-methyltetrahydrofuran, diethyl ether, dioxane, 1,4-dioxane, 1,2-dioxane, 1,3-dioxane, and combinations thereof; an alcoholic solvent selected from the group consisting of methanol, ethanol, isopropanol, t-amyl alcohol, t-butyl alcohol, hexanol, and combinations thereof; a halogenated solvent selected from the group consisting of dichloromethane, 4-bromotoluene, diiodomethane, carbon tetrachloride, chlorobenzene, chloroform, and combinations thereof; a ketone solvent selected from the group consisting of acetone, propanone, methyl ethyl ketone, methyl isobutyl ketone, and combinations thereof; an aprotic solvent selected from the group consisting of acetonitrile, N,N-dimethyl formamide (DMF), N,N-dimethyl acetamide, dimethyl sulfoxide (DMSO), N-methylpyrrolidone (NMP), and combinations thereof; an aromatic solvent selected from the group consisting of toluene, xylene, benzene, and combinations thereof; water; or a mixture thereof.

3. The process as claimed in claim 1, wherein the base is an alkali metal hydroxide selected from the group consisting of lithium hydroxide, sodium hydroxide, potassium hydroxide; alkali metal carbonate selected from the group consisting of lithium carbonate, sodium carbonate, potassium carbonate, cesium carbonate, and combinations thereof; alkali metal bicarbonates selected from the group consisting of sodium bicarbonate, potassium bicarbonate, and combinations thereof; alkali metal alkoxides selected from the group consisting of sodium methoxide or potassium methoxide, sodium ethoxide or potassium ethoxide, potassium tert-butoxide, and combinations thereof: organic amines selected from the group consisting of triethylamine, diisopropylethylamine, pyridine, 1,8-diazabicyclo[5.4.0]undec-7-ene (DBU) or 1,5-diazabicyclo[4.3.0]non-5-ene (DBN), and combinations thereof.

4. The process as claimed in claim 3, wherein the base is an aqueous base selected from the group consisting of sodium hydroxide, potassium hydroxide, barium hydroxide, lithium hydroxide, sodium methoxide, sodium ethoxide, potassium tert butoxide, potassium carbonate, sodium carbonate, cesium carbonate, sodium bicarbonate, potassium bicarbonate, and combinations thereof.

5. The process as claimed in claim 1, wherein Levoketoconazole (1) is isolated and purified from a reaction mass of step (b), comprising the steps of:
(i) concentrating the reaction mass of step (b) to obtain a residue;
(ii) crystallizing the obtained residue of step (i) in a solvent to form a precipitate; and
(iii) filtering the precipitate of step (ii) and drying the obtained precipitate to obtain crystalline solid of Levoketoconazole (1).

6. The process as claimed in claim 5, wherein the solvent used in step (ii) is an alcoholic solvent selected from methanol, ethanol, isopropanol, t-amyl alcohol, t-butyl alcohol or hexanol.

7. Crystalline Levoketoconazole, characterized by a powder X-Ray diffraction pattern having peaks at 10.51, 15.95, 17.42, 19.27, 19.87, 21.15 and 23.62±0.2 degrees of 2-theta.

8. The crystalline Levoketoconazole as claimed in claim 7, having an endotherm between 159° C. to 162° C. in a differential scanning calorimetric (DSC) thermogram.

9. The crystalline Levoketoconazole as claimed in claim 7, being anhydrous in nature.

10. The crystalline Levoketoconazole as claimed in claim 7 having a purity of >99.85% as determined by HPLC.

11. The process as claimed in claim 1, wherein the pure Levoketoconazole has a purity of >99.85% as determined by HPLC.
